(19) United States
(12) Patent Application Publication
(10) Pub. No.: US 2019/0338257 A1
(43) Pub. Date: Nov. 7, 2019

(54) ELECTRON TRANSPORT CHAIN MODULE FROM EUKARYOTIC ORGANELLE AND APPLICATION THEREOF

(71) Applicant: PEKING UNIVERSITY, Beijing (CN)

(72) Inventors: Jianguo YANG, Beijing (CN); Mingxuan YANG, Beijing (CN); Ray DIXON, Beijing (CN); Yiping WANG, Beijing (CN); Xiaqing XIE, Beijing (CN)

(73) Assignee: PEKING UNIVERSITY, Beijing (CN)

(21) Appl. No.: 16/475,646

(22) PCT Filed: Dec. 22, 2017
(86) PCT No.: PCT/CN2017/117862

§ 371 Date: Jul. 2, 2019

(30) Foreign Application Priority Data
Jan. 3, 2017 (CN) 201710001952.9

(51) Int. Cl. C12N 9/02; C07K 14/415; C05G 3/08

(52) U.S. Cl. CPC C12N 9/0095; C07K 14/415; C05G 3/08; C12Y 118/01006; C12Y 118/01003

(57) ABSTRACT

Provided are an electron transport chain module from a eukaryotic organelle and an application thereof in biological nitrogen fixation. The electron transport chain (ETC) module is composed of the NifJ protein from Klebsiella oxytoca and a ferredoxin from plant chloroplasts or leucoplasts; plant-type ferredoxin-NADPH reductase (FNR) and the FdxH or FdxB protein from Anabaena; or an FNR and a Ferredoxin protein from plant organelles.

### TECHNICAL FIELD

The present invention relates to an electron transport chain (ETC) module from eukaryotic organelles and application of the ETC module in biological nitrogen fixation.

### BACKGROUND ART

Nitrogen is one of the primary nutrients limiting crop growth and yield in agriculture^{[1]}. The use of industrial nitrogen fertilizer can circumvent this limitation and provide sufficient nitrogen source for crop growth. However, the extensive use of industrial nitrogen fertilizers can lead to environmental problems, and the economic costs of nitrogen fertilizers used are relatively high. These problems are particularly significant in developing countries^{[2-3]}. These factors have led researchers to refocus on reconstructing the biological nitrogenase system in crops by engineering methods to achieve crop self-nitrogen fixation to solve the problem of nitrogen fertilizer use. Biological nitrogen fixation (BNF), a process that converts gaseous nitrogen to ammonia by nitrogenases in diazotrophs, contributes over 60% of the nitrogen in the atmospheric nitrogen cycle^{[4]}. Nitrogenases are a family of metalloenzymes that consist of two separable components, dinitrogenase reductase (Fe protein) and dinitrogenase (XFe protein, where X is Mo, V, or Fe, depending on the metal atom composition of the active site cofactor) (see FIG. 1)^{[5-6]}. The process of all three nitrogenase-catalyzed reduction of N₂ can be summarized as the following equation: N₂+(6+2n)H⁺+(6+2n)e⁻→2NH₃+nH₂ (n≥1)^{[7-9]}. In this process, electrons are first transferred to the Fe protein, which in turn donates electrons to the XFe protein with hydrolysis of two molecules of ATP per electron^{[10-11]}. Although Fe protein is the obligate electron donor for XFe protein in all nitrogenase systems, the electron donor for Fe protein in diazotrophs is not conserved^{[9]}. Direct electron donors to Fe protein are either reduced flavodoxin or reduced ferredoxin, which depends on the physiology of the host diazotroph^{[13-17]}.

A number of studies have suggested chloroplasts, root-plastids or mitochondria can be ideal locations to introduce a nitrogenase system in eukaryotes^{[18-20]}. These organelles responsible for energy conversion can potentially provide reducing power and ATP required for nitrogen fixation process. Diverse reduction reactions carried out in these organelles rely on different electron transport chains (ETCs)^{[21].} Existing researches have confirmed that multiple copies of ferredoxin are contained in plant cells, including photosynthetic or non-photosynthetic ferredoxins expressed in chloroplasts or root-plastids; and mitochondria ferredoxin-like adrenodoxins (MFD) located in the mitochondria^{[21-22]}. The major function of the photosynthetic ferredoxins expressed in chloroplasts is to mediate the transfer of electrons from photosystem I (PSI) to Ferredoxin-NADPH oxidoreductase (leaf-type FNR, LFNR) to catalyze the production of NADPH^{[23]}. In addition, photosynthetic ferredoxins are also responsible for the distribution of reducing power derived from the photosynthetic process to proteins involved in nitrogen and sulfur assimilation^{[24]}. Electron transfer between root-type FNR (RFNR) and ferredoxin in the root-plastid is opposite to that in the chloroplast, with NADPH generated in the oxidative pentose-phosphate pathway (OxPPP) transferring electrons to ferredoxin via RFNR to reduce the ferredoxin protein^{[25]}. In mitochondria, MFD mediates the transfer of electrons from NADPH-dependent adrenodoxin oxidoreductase (MFDR) to the cysteine desulfurase Nfs1 to participate in the biosynthesis of the biotin^{[26]}.

In the previous study, we have successfully constructed recombinant MoFe^{[27]} nitrogenase systems from Klebsiella oxytoca (Ko) and the “minimal” FeFe^{[28]} nitrogenase systems from Azotobacter vinelandii (Av) in Escherichia coli (FIG. 1). From the synthetic biology and systemic biology viewpoints, these two nitrogenase systems can be divided into three functional modules in the present invention: the ETC module, the metal cluster biosynthesis module and the “core” enzyme module (FIG. 1). In turn, we investigated whether the ETC module from plant plastids and mitochondria provides the reducing power required for nitrogen reduction for the “core” enzyme module of the nitrogenase system (including MoFe or FeFe nitrogenase system) by using E. coli as a “chassis”. Our results indicate that intact ETC modules from the chloroplast and root-plastid, or hybrid modules from plastid or mitochondria can functionally support nitrogenase activity. Therefore, our research solves the problem of electron transport chain module selection when engineering a nitrogenase system in different plant organelles.

### SUMMARY OF THE INVENTION

Biological nitrogen fixation is a complex system involving many genes, and it is also a process that requires a large amount of ATP and reducing power. Thus, the involvement of excess genes in the nitrogenase system, the energy available to the nitrogenase system in a particular host environment, and the reducing power are major bottlenecks in the reconstitution of nitrogenase systems in crops. In recent studies, attempts have been made to reduce the number of structural genes required for MoFe nitrogenase^{[29]} and FeFe nitrogenase^{[28]} to simplify the nitrogenase system. However, it was found that when the number of structural genes was reduced to 9, the activity of MoFe nitrogenase^{[29]} decreased sharply, and when some genes were replenished into the system, the nitrogenase activity could be restored to a higher level^{[30]}. These results demonstrate the difficulty of simplifying the nitrogenase system without losing the efficiency of nitrogenase.

In the present invention, we first introduced the concept of modularization of synthetic biology and systematic biology, and divided the nitrogenase system into three functionally independent modules: an electron transport chain (ETC) module, a metal cluster synthesis module, and a nitrogenase “core” enzyme module. And it was further investigated that if electron transport components from plant chloroplasts, root-plastids or mitochondria, including ferredoxin or Ferredoxin-NADPH oxidoreductase (FNR), can replace NifF or NifJ proteins in ETC modules, respectively, to provide electrons for the MoFe and FeFe nitrogenase systems. In this study, we used the model organism E. coli as the “chassis” to study the compatibility of the “core” enzyme module of the FeFe/MoFe nitrogenase system with ETC modules from plant organelles such as chloroplasts, root-plastids and mitochondria.

In the present invention, a new ETC module in which the NifJ or/and NifF protein is replaced is recombinantly produced, and whether the plant-derived ETC module can substitute the NifJ or NifF protein in the ETC module to provide electrons for the “core” enzyme module of the FeFe/MoFe nitrogenase system to support the activity of nitrogenases is determined by the acetylene reduction method and the ¹⁵N₂ assimilation assay. The means of “substitution” described herein include: the NifF protein is replaced by the ferredoxin protein derived from plant organelles, or the NifJ protein is replaced by the FNR protein derived from plant organelles, or both of the above replacements are performed simultaneously. The ETC module formed when one of the NifJ or NifF proteins is replaced, is called a hybrid module, and the ETC module formed when they are simultaneously replaced by the ferredoxin protein and the FNR protein of the plant organelle, is called an intact module. More specifically, a hybrid ETC module described herein is formed by replacing NifF in the NifJ-NifF module with ferredoxin from chloroplasts, root-plastids or mitochondria of various representative plants, or is composed by the hybrid ETC module consisting of plant-type FNR and Anabaena sp. PCC 7120 (As) FdxH or FdxB. The intact ETC module described herein is a plant-derived ETC module formed by replacing the NifJ and NifF proteins present in the ETC module of diazotrophs with encoded ferredoxin-NADPH oxidoreductase (FNR) and ferredoxin from the target plant organelle, respectively.

The results of the present study indicate that all plant-derived ferredoxins except ferredoxins from mitochondria can functionally replace NiFe of FeFe and MoFe nitrogenases, which means that the interaction between these ferredoxins and NifH/AnfH can meet the needs of electron transport; the intact ETC module (FNR-Ferredoxin) from the chloroplasts and root-plastids of various plants can support the activity of nitrogenase, which means that engineering and reconstituting nitrogen fixation system in the plant plastid does not need to additionally carry the ETC module; the hybrid module formed by mitochondrial MFDR and Anabaena FdxH/FdxB can support the nitrogenase activity; and after analyzing the source of the above substitution components, it can be concluded that the chloroplast-derived ETC module can provide the most suitable electron supply for the nitrogenase system in E. coli.

Therefore, based on the technical solution of the above-mentioned replaceable ETC module, it is beneficial for us to use the endogenous ETC derived from plant organelles in the process of biological nitrogen fixation in the future, thereby avoiding the technical obstacles caused by excessive number of nitrogenase structural genes, high energy demand and reducing power in the process of engineering and reconstituting the biological nitrogenase system in plant cells.

More specifically, the present invention specifically relates to the following aspects:

One aspect of the invention relates to an electron transport chain (ETC) module for a nitrogen fixation system, comprising an NifJ protein and an NifF protein.

The ETC module described in the above aspect, wherein the nitrogen fixation system is MoFe nitrogenase system and FeFe nitrogenase system.

The ETC module described in the above aspects, wherein the NifJ and NifF proteins are substituted individually or substituted simultaneously by corresponding proteins from eukaryotic organelles, thereby forming a hybrid or intact ETC module.

The ETC module described in the above aspects, wherein the eukaryotic organism is a plant, and the organelle is a plastid or mitochondria.

The ETC module described in the above aspects, wherein the hybrid ETC module is formed by replacing the NifF protein in the ETC module by ferredoxins from a plant plastid.

The ETC module described in the above aspects, wherein the plastid is a chloroplast or a root-plastid, preferably a chloroplast.

The ETC module described in the above aspects, wherein the hybrid ETC module is formed by replacing an NifJ protein in the ETC module consisting of the NifJ and the NifF by plant-type ferredoxin-NADPH reductase (FNR) from a plant plastid and mitochondria.

The ETC module described in the above aspects, wherein the plastid is a chloroplast or a root-plastid.

The ETC module described in the above aspects, wherein the hybrid ETC module is composed of NADPH-dependent adrenodoxin oxidoreductase (MFDR) from plant mitochondria and Anabaena FdxB.

The ETC module described in the above aspects, wherein the intact ETC module is composed of FNRs from target plant organelles and ferredoxin proteins.

The ETC module described in the above aspects, wherein the target plant organelle is a chloroplast or a root-plastid.

The use of the ETC module of any of the preceding items in biological nitrogen fixation.

### DESCRIPTION OF THE DRAWINGS

FIG. 1. Upper: Modular arrangement of the recombined MoFe and the “minimal” FeFe nitrogenase system. Letters in the diagram represent the corresponding nif or anfHDGK genes, e.g. J represents nifJ gene. Bottom: Schematic diagram of electron transport within nitrogenase in a nitrogenase system, with the representative proteins in figure shown with crystal structures, wherein the abbreviation PFO (NifJ) represents pyruvate-ferredoxin (Flavodoxin) oxidoreductase; the abbreviation FNR represents ferredoxin-NADPH oxidoreductase; the abbreviation NifF represents flavodoxin; the abbreviation FdxN represents 2[4Fe-4S]-type ferredoxin; the abbreviation FdxH represents [2Fe-2S]-type ferredoxin; the abbreviation Fe protein represents dinitrogenase reductase; X=Mo, V or Fe in XFe protein. The cofactors of the Fe proteins and XFe proteins are shown in ball-and-stick model. Atom colors are Fe in rust, S in yellow, C in gray, 0 in red and X atom (Mo, V or Fe atom) in purple. Additionally, gene arrangement in this figure does not represent the real gene arrangement for the recombined MoFe and the “minimal” FeFe nitrogenase system.

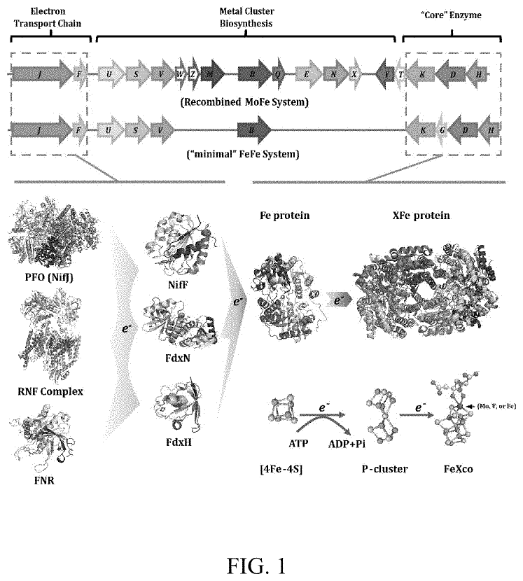

FIG. 2. (A) Sequence alignment of the AsFdxH protein with ferredoxins from plastids. (B) Sequence alignment of the AsFdxH protein with ferredoxins from mitochondria. In the figure, sequences in green shadow in (A) or crimson shadow in (B) are leading peptides for the plant-type ferredoxins; cysteine residues for binding [2Fe-2S] are highlighted with yellow shadow. As represents Anabaena sp. PCC 7120; Cr represents Chlamydomonas reinhardtii; At represents Arabidopsis thaliana; Zm represents Zea mays; Os represents Oryza sativa; Ta represents Triticum aestivum.

FIG. 3. Plasmid maps of the main vectors used in the invention. The rrnB T1 is a Escherichia coli terminator (BBa_B0010); the T_{L} is the E. coli thrL gene terminator; the T₀ is a phage lambda t₀ terminator; the Tₐ is an artificial terminator L3S2P21 reported by chen et al^{[1]}.

FIG. 4. Gradient induction assays of the controllable expression of the CrPETF or CrFNR. (A) and (B) Gradient induction of the expression of the CrPETF controlled by the P_{LtetO-1} promoter with the anhydrotetracycline (aTc). (C) and (D) Gradient induction of the expression of the CrFNR controlled by the Ptac promoter with the isopropyl-β-d-thiogalactoside (IPTG). In this experiment, the expression of CrPETF was first induced with 200 ng/mL of aTc, and then the expression of CrFNR was induced with gradient concentration of IPTG. The acetylene reduction activities shown in the figure were relative activities obtained under the condition that the activities obtained from FeFe or MoFe nitrogenase system carrying NifJ-NifF module are defined as 100% activities. FeFe represents the “minimal” FeFe nitrogenase system; MoFe represents the recombined MoFe nitrogenase system. Error bars indicate the standard deviation observed from at least three independent experiments.

FIG. 5. Substitution effect of the hybrid ETC modules consisting of the NifJ protein and ferredoxin from plastid was assayed by acetylene reduction. (A) Schematic picture of electron transport pathways between the hybrid ETC modules and the “core” enzyme module. NifJ-NifF module was replaced by the hybrid ETC modules comprising the NifJ with chloroplast ferredoxins (B and C); NifJ with root-plastid ferredoxins (D and E); NifJ with mitochondria ferredoxins (F and G). The activities obtained from FeFe or MoFe nitrogenase system carrying NifJ-NifF module without adding inducers are defined as 100%. FeFe, represents the “minimal” FeFe nitrogenase system; MoFe, represents the recombined MoFe nitrogenase system. As represents Anabaena sp. PCC 7120; Cr represents Chlamydomonas reinhardtii; At represents Arabidopsis thaliana; Zm represents Zea mays; Os represents Oryza sativa; Ta represents Triticum aestivum. Error bars indicate the standard deviation observed from at least three independent experiments.

FIG. 6. MoFe or FeFe nitrogenase activities after a high-copy plasmid carrying the GroESL-encoding genes was cotransformed with NifJ-AtMFD1 or NifJ-AtMFD2 hybrid modules were assayed by acetylene reduction. The activities obtained from FeFe or MoFe nitrogenase system carrying NifJ-NifF module without adding inducers are defined as 100%. FeFe, represents the “minimal” FeFe nitrogenase system; MoFe, represents the recombined MoFe nitrogenase system. At represents Arabidopsis thaliana. Error bars indicate the standard deviation observed from at least three independent experiments.

FIG. 7. Substitution effect of the hybrid ETC modules consisting of the KoNifF or AsFdxB with FNRs from different plant organelles was assayed by acetylene reduction. The NifJ-NifF module was replaced by the hybrid modules consisting of plant-type FNRs and the KoNifF (A and B) or AsFdxB (C and D). The acetylene reduction activities obtained from FeFe or MoFe nitrogenase system carrying NifJ-NifF module without adding inducers are defined as 100%. FeFe, represents the “minimal” FeFe nitrogenase system; MoFe, represents the recombined MoFe nitrogenase system. Ko represents Klebsiella oxytoca; As represents Anabaena sp. PCC 7120; Cr represents Chlamydomonas reinhardtii; At represents Arabidopsis thaliana; Zm represents Zea mays. Error bars indicate the standard deviation observed from at least three independent experiments.

FIG. 8. Substitution effect of the hybrid ETC modules consisting of the plant FNRs and AsFdxH was assayed by acetylene reduction. (A) Schematic picture of electron transport pathways between the hybrid ETC modules and the “core” enzyme module. The NifJ-NifF module was replaced by the hybrid modules consisting of the plant FNRs and AsFdxH (B and C). The activities obtained from FeFe or MoFe nitrogenase system carrying NifJ-NifF module without adding inducers are defined as 100%. FeFe, represents the “minimal” FeFe nitrogenase system; MoFe, represents the recombined MoFe nitrogenase system. As, Anabaena sp. PCC 7120; Cr, Chlamydomonas reinhardtii; At, Arabidopsis thaliana; Zm, Zea mays. Error bars indicate the standard deviation observed from at least three independent experiments.

FIG. 9. Substitution effect of the intact ETC modules of chloroplast and root-plastid was assayed by acetylene reduction and ¹⁵N assimilation assays (D and E). (A) Schematic picture shows electron transport pathways between the intact ETC modules from plant organelles and the “core” enzyme module. The activities obtained from FeFe or MoFe nitrogenase system carrying NifJ-NifF module without adding inducers are represented by 100%. FeFe, represents the “minimal” FeFe nitrogenase system; MoFe, represents the recombined MoFe nitrogenase system. As, Anabaena sp. PCC 7120; Cr, Chlamydomonas reinhardtii; At, Arabidopsis thaliana; Zm, Zea mays. Error bars for the acetylene reduction assay indicate the standard deviation observed from at least three independent experiments. Error bars for the ¹⁵N assimilation assay indicate the standard deviation observed from at least two independent experiments.

FIG. 10. Single component of the intact ETC module is not enough to support nitrogenase activity for the FeFe (A) or MoFe (B) nitrogenase system. The acetylene reduction activities obtained from FeFe or MoFe nitrogenase system carrying NifJ-NifF module without adding inducers are defined as 100%. FeFe, represents the “minimal” FeFe nitrogenase system; MoFe, represents the recombined MoFe nitrogenase system. As, Anabaena sp. PCC 7120; Cr, Chlamydomonas reinhardtii; At, Arabidopsis thaliana; Zm, Zea mays. Error bars indicate the standard deviation observed from at least three independent experiments.

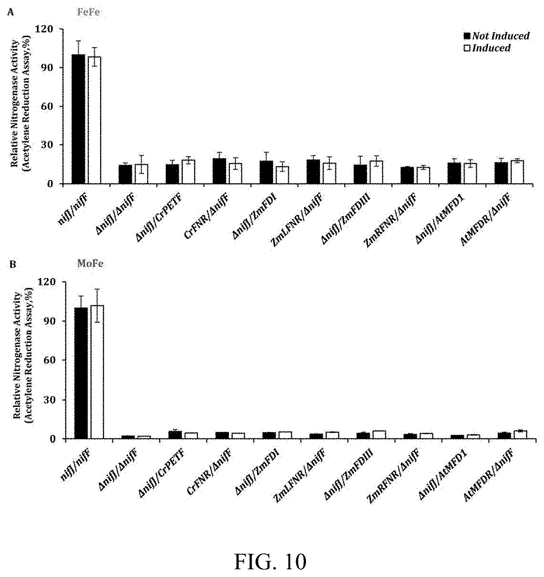

### EXAMPLES

Materials and Methods

Bacterial Strains and Plasmids Used in the Examples are Shown in Table 1

TABLE 1
Bacterial Strains and plasmids used in the Examples

| Strain or | | Source or |
|---|---|---|
| plasmid | Relevant feature | reference |
| E. coli Strains | | |
| Top 10 | F⁻ mcrA Δ(mrr-hsdRMS-mcrBC) φ80lacZΔM15 | Invitrogen |
| | ΔlacX74 nupG recA1 araD139 Δ(ara-leu)7697 | |
| | galE15 galK16 rpsL(Str^{R}) endA1 λ⁻ | |
| JM109 | recA endA1 gyrA96 hsdR17 supE44 relA1 | Takara |
| | Δ(lac-proAB)/F′[traD36 proAB⁺ lacI^{q} lacZΔM15] | Bio |
| Plasmids | | |
| pBR322M | pBR322 derivative, Amp^{R}, labeled as ΔnifJ/ΔnifF in | (8) |
| | the text | |
| pBR322M-P_{LtetO-1} | pBR322M derivative carrying P_{LtetO-1} inducible | Examples |
| | expression cassette | |
| pBR322M-P_{tac} | pBR322M derivative carrying P_{tac} inducible | Examples |
| | expression cassette | |
| pKU7815 | pACYC184 derivative carrying the “minimal” FeFe | (8) |
| | nitrogenase system, Cm^{R} | |
| pKU7017 | pACYC184 derivative carrying the recombined | (9) |
| | MoFe nitrogenase system, Cm^{R} | |
| pKU7830 | pKU7815 derivative, ΔnifJ/ΔnifF | Examples |
| pKU7831 | pKU7017 derivative, ΔnifJ/ΔnifF | Examples |
| pKU7832 | pBR322M derivative carrying KonifJ/nifF genes, | Examples |
| | labeled as nifJ/nifF in the text | |
| pKU7833 | pKU7830 derivative with nifF gene replaced by | Examples |
| | P_{LtetO-1} inducible expression cassette, labeled as | |
| | nifJ/ΔnifF in the text | |
| pKU7834 | pKU7833 derivative carrying nifJ/P_{LtetO-1}-AsfdxHₒᵣᵢ | Examples |
| pKU7835 | pKU7833 derivative carrying nifJ/P_{LtetO-1}-CrPETF_{syn} | Examples |
| pKU7836 | pKU7833 derivative carrying nifJ/P_{LtetO-1}-AtFD2_{syn} | Examples |
| pKU7837 | pKU7833 derivative carrying nifJ/P_{LtetO-1}-ZmFDI_{syn} | Examples |
| pKU7838 | pKU7833 derivative carrying nifJ/P_{LtetO-1}-OsFD1_{syn} | Examples |
| pKU7839 | pKU7833 derivative carrying nifJ/P_{LtetO-1}-TaFD_{syn} | Examples |
| pKU7840 | pKU7833 derivative carrying nifJ/P_{LtetO-1}-AtFD3_{syn} | Examples |
| pKU7841 | pKU7833 derivative carrying nifJ/P_{LtetO-1}-ZmFDIII_{syn} | Examples |
| pKU7842 | pKU7833 derivative carrying nifJ/P_{LtetO-1}-OsFD4_{syn} | Examples |
| pKU7843 | pKU7833 derivative carrying nifJ/P_{LtetO-1}-AtMFD1_{syn} | Examples |
| pKU7844 | pKU7833 derivative carrying nifJ/P_{LtetO-1}-AtMFD2_{syn} | Examples |
| pKU7845 | pEASY-Blunt derivative carrying EcgroES operon | Examples |
| | with its original promoter | |
| pKU7846 | pKU7832 derivative with nifJ gene replaced by P_{tac} | Examples |
| | inducible expression cassette, labeled as ΔnifJ/nifF | |
| | in the text | |
| pKU7847 | pKU7846 derivative carrying P_{tac}-AspetHₒᵣᵢ/nifF | Examples |
| pKU7848 | pKU7846 derivative carrying P_{tac}-CrFNR_{syn}/nifF | Examples |
| pKU7849 | pKU7846 derivative carrying P_{tac}-ZmLFNR_{syn}/nifF | Examples |
| pKU7850 | pKU7846 derivative carrying P_{tac}-ZmRFNR_{syn}/nifF | Examples |
| pKU7851 | pKU7846 derivative carrying P_{tac}-AtMFDR_{syn}/nifF | Examples |
| pKU7852 | pKU7834 derivative with nifJ gene replaced by P_{tac} | Examples |
| | inducible expression cassette, labeled as ΔnifJ/ | |
| | P_{LtetO-1}-AsfdxHₒᵣᵢ in the text | |
| pKU7853 | pKU7847 derivative carrying P_{tac}-AspetHₒᵣᵢ/ | Examples |
| | P_{LtetO-1}-AsfdxHₒᵣᵢ | |
| pKU7854 | pKU7848 derivative carrying P_{tac}-CrFNR_{syn}/ | Examples |
| | P_{LtetO-1}-AsfdxHₒᵣᵢ | |
| pKU7855 | pKU7849 derivative carrying P_{tac}-ZmLFNR_{syn}/ | Examples |
| | P_{LtetO-1}-AsfdxHₒᵣᵢ | |
| pKU7856 | pKU7850 derivative carrying P_{tac}-ZmRFNR_{syn}/ | Examples |
| | P_{LtetO-1}-AsfdxHₒᵣᵢ | |
| pKU7857 | pKU7851 derivative carrying P_{tac}-AtMFDR_{syn}/ | Examples |
| | P_{LtetO-1}-AsfdxHₒᵣᵢ | |
| pKU7858 | pKU7833 derivative carrying nifJ/P_{Lteto-1}-AsfdxBₒᵣᵢ | Examples |
| pKU7859 | pKU7847 derivative carrying P_{tac}-AspetHₒᵣᵢ/ | Examples |
| | P_{LtetO-1}-AsfdxBₒᵣᵢ | |
| pKU7858 | pKU7848 derivative carrying P_{tac}-CrFNR_{syn}/ | Examples |
| | P_{LtetO-1}-AsfdxBₒᵣᵢ | |
| pKU7859 | pKU7849 derivative carrying P_{tac}-ZmLFNR_{syn}/ | Examples |
| | P_{LtetO-1}-AsfdxBₒᵣᵢ | |
| pKU7860 | pKU7850 derivative carrying P_{tac}-ZmRFNR_{syn}/ | Examples |
| | P_{LtetO-1}-AsfdxBₒᵣᵢ | |
| pKU7861 | pKU7851 derivative carrying P_{tac}-AtMFDR_{syn}/ | Examples |
| | P_{LtetO-1}-AsfdxBₒᵣᵢ | |
| pKU7862 | pKU7848 derivative carrying P_{tac}-CrFNR_{Syn}/ | Examples |
| | P_{LtetO-1}-CrPETF_{syn} | |
| pKU7863 | pKU7849 derivative carrying P_{tac}-ZmLFNR_{syn}/ | Examples |
| | P_{LtetO-1}-ZmFDI_{syn} | |
| pKU7864 | pKU7850 derivative carrying P_{tac}-ZmRFNR_{syn}/ | Examples |
| | P_{LtetO-1}-ZmFDIII_{syn} | |
| pKU7865 | pKU7851 derivative carrying P_{tac}-AtMFDR_{syn}/ | Examples |
| | P_{LtetO-1}-AtMFD_{syn} | |
| pKU7866 | pKU7833 derivative carrying nifJ/P_{LtetO-1}-AspetFₒᵣᵢ | Examples |
| pKU7867 | pKU7833 derivative carrying nifJ/P_{LtetO-1}-CrFDX2_{syn} | Examples |
| pKU7868 | pKU7833 derivative carrying nifJ/P_{LtetO-1}-AtFD1_{syn} | Examples |
| pKU7869 | pKU7833 derivative carrying nifJ/P_{LtetO-1}-ZmFDII_{syn} | Examples |

Bacterial Strains and Growth Medium

Luria-Bertani (LB) broth for E. coli growth contained 10 g/L of Tryptone, 5 g/L of Yeast Extract and 10 g/L of NaCl. KPM minimal media used in this study contained 10.4 g/L of Na₂HPO₄, 3.4 g/L of KH₂PO₄, 26 mg/L of CaCl₂. 2H₂O, 30 mg/L of MgSO₄, 0.3 mg/L of MnSO₄, 36 mg/L of ferric citrate, 10 mg/L of para-aminobenzoic acid, 5 mg/L of biotin, 1 mg/L Vitamin B1 and 0.8% (w/v) glucose, with 20 mM ammonium salt (KPM-HN) or 10 mM glutamate (KPM-LN) as the nitrogen source. Casamino acids (purchased from BD Biosciences, 223050) at final concentration of 0.05% were also added to the KPM minimal media to ensure the normal growth. Antibiotics were used at the following concentration: 50 μg/mL for ampicillin, 25 μg/mL for chloramphenicol.

Construction of Recombinant Plasmids

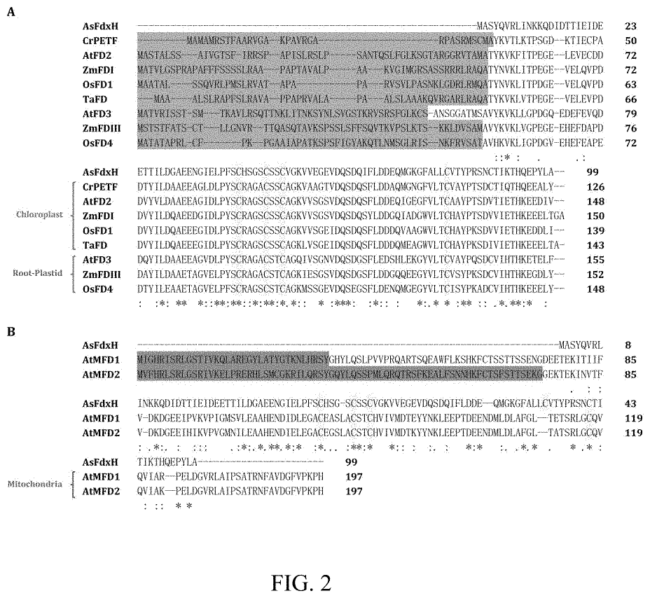

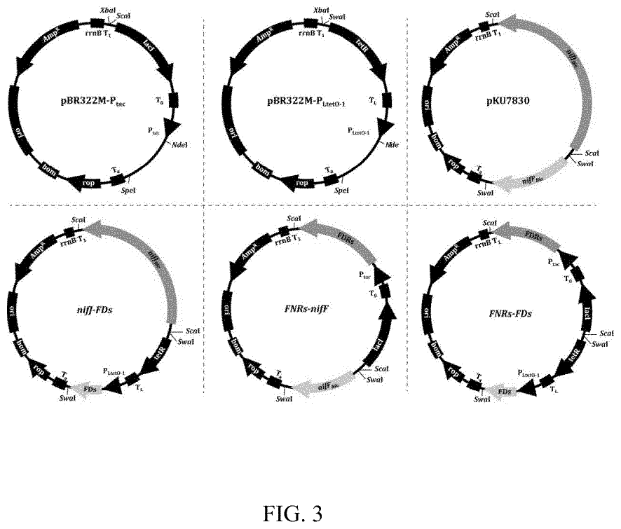

Plasmid pKU7815 and pKU7017 are pACYC184 derivatives carrying the “minimal” FeFe^{[28]} or the recombined MoFe^{[27]} nitrogenase system. nifF and nifJ double deletion derived plasmid of pKU7815 and pKU7017, designed as pKU7830 and pKU7831 in the Examples, were constructed by direct removal of the nifF and nifJ operons using the specific restriction sites flanking each operon, SwaI for the nifF and ScaI for the nifJ respectively. The complementary plasmid for the pKU7830 and pKU7831 is a pBR322M derived plasmid (pKU7832) carrying the nifF and nifJ genes. The pBR322M-P_{LtetO-1} plasmid was obtained by direct reassembly of the tetR expression cassette and the P_{LtetO-1} promoter region with the pBR322M as backbone using Gibson Assemble kit (NEB, E5520S). The tetR expression cassette comprises a strong constitutive promoter (BBa_J23100, https://parts.igem.org), a medium ribosome binding site (RBS, BBa_B0032, https://parts.igem.org) and thrL terminator from E. coli. Similarly, the pBR322M-P_{tac} plasmid was obtained by direct reassembly of the lad expression cassette and the P_{tac} promoter carrying a weak RBS with pBR322M as backbone. To lower the leakage expression of the plant-type FNRs, a Lad mutant LacI^{WF[31]} with higher affinity for the lac operator was used for construction of pBR322M-P_{tac} plasmid. The nifF gene in the pKU7832 was replaced with the SwaI restricted fragment [tetR-P_{LtetO-1}] fragment from plasmid pBR322M P_{LtetO-1}, resulting in plasmid pKU7833. To construct the plasmid for expression of the hybrid modules consisting of the NifJ and plant-type ferredoxins, original ferredoxin gene sequences or chemically synthesized ferredoxin gene sequences were cloned downstream of the P_{LtetO-1} promoter of the pKU7833 plasmid by using NdeI/SpeI restriction sites. In order to facilitate detection of the expression level of different ferredoxins, a sequence encoding the Histidine-tag was added to each of the synthesized ferredoxin sequence as shown in gray shadow in FIG. 2. The nifJ gene in the pKU7832 was replaced with the ScaI restricted fragment [lacI-P_{tac}] fragment from plasmid pBR322M-P_{tac}, resulting in plasmid pKU7846. To construct the plasmid expressing the hybrid modules consisting of the plant-type FNRs and NifF, original FNR gene sequences or chemically synthesized FNR gene sequences were cloned downstream of the P_{tac} promoter of the pKU7846 plasmid by using NdellSpeI restriction sites. The pKU7853 plasmid, carrying P_{tac}-AspetHₒᵣᵢ/P_{LtetO-1}-AsfdxHₒᵣᵢ, was constructed by replacing the nifF gene in plasmid pKU7847 with ScaI restricted fragment [P_{LtetO-1}-AsfdxH] fragment from pKU7834. Similar methods were used to construct the pKU7854-pKU7857 and pKU7859-pKU7865. The PCR product carrying the groES with its original promoter, flanking the XbaI/SpeI restriction sites, was PCR amplified from the genome of E. coli and directly ligated to the pEASY-Blunt vector (TransGene, CB101) to generate pKU7845. Plasmids maps are provided in FIG. 3. Each of the above constructed plasmids was confirmed by DNA sequencing before any further experiments.

Acetylene Reduction Assay

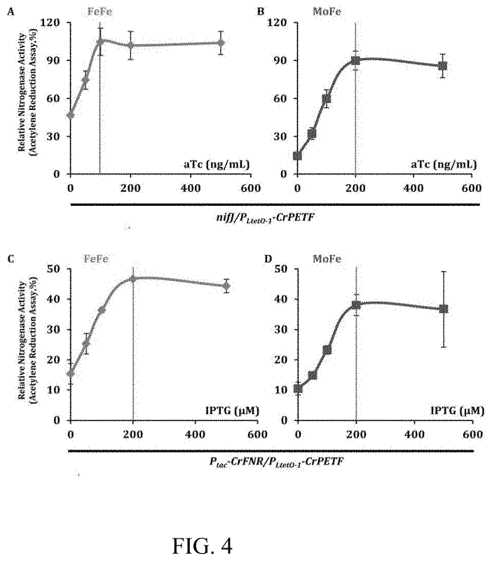

The C₂H₂ reduction method was used to assay the nitrogenase activity as described in the literature^{[32]}. To measure nitrogenase activity of the recombined E. coli JM109 strains, cells were initially grown overnight in KPM-HN medium. The cells were then diluted into 2 mL KPM-LN medium in 20 mL sealed tubes to a final OD₆₀₀ of ˜0.3. In order to maximize the restoring effect for the plant-type ferredoxins and FNRs, 200 ng/mL of anhydrotetracycline (aTc) or 200 μM of isopropyl-β-d-thiogalactoside (IPTG) was added to induce the expression of the ferredoxins or FNRs respectively as indicated by results shown in FIG. 4. Air in the tube was repeatedly evacuated and flushed with argon three times. After static culture at 30° C. for 6˜8 h, 2 mL C₂H₂ was added. The activity was detected ˜16 h later with a Shimadzu GC-2014 gas chromatograph. Data presented are mean values based on at least three replicates.

¹⁵N₂ Assimilation Assay

To detect the ¹⁵N₂ assimilation activity, the recombined E. coli JM109 strains were prepared as described in the acetylene reduction assay. Air in the tube was repeatedly evacuated and flushed with nitrogen three times. 3 mL gas was finally removed and 2 mL ¹⁵N₂ gas (99%⁺, Shanghai Engineering Research Center for Stable Isotope) was injected. After 48 h of incubation at 30° C., the cultures were collected, and were freeze dried, ground, weighed and sealed into tin capsules. Isotope ratios are expressed as δ¹⁵N whose values are a linear transform of the isotope ratios, ¹⁵N/¹⁴N representing the per mille difference between the isotope ratios in a sample and in atmospheric N₂^{[33]}. Data presented are mean values based on at least two replicates.

### Example 1: Hybrid ETC Modules Consisting of the NifJ Protein and Plastid Ferredoxins can Functionally Support Nitrogenase Activity

Most plants are known to have multiple copies of ferredoxins in different organelles^{[21]}. Through preliminary sequence analysis, we found that ferredoxins from plant chloroplast and root-plastid show high sequence identity with the Anabaena sp. PCC 7120 (As)fdxH gene product, which is the primary electron donor for the nitrogenase in the cyanobacteria^{[34]}. To investigate if hybrid ETC modules formed by the NifJ protein and plastid ferredoxins could support nitrogenase activity in E. coli. Coding sequences of several representative plastid ferredoxins from Chlamydomonas reinhardtii (Cr), Arabidopsis thaliana (At), Zea mays (Zm), Oryza sativa (Os), and Triticum aestivum (Ta) were selected for further study. These selected representative ferredoxin encoding genes were codon optimized according to the codon bias of E. coli, optimized gene sequence shown in Sequence Listing, and expressed from the inducible P_{Lteto-1} promoter. The fdxH gene from As was used as a control to verify effectiveness of the inducible system.

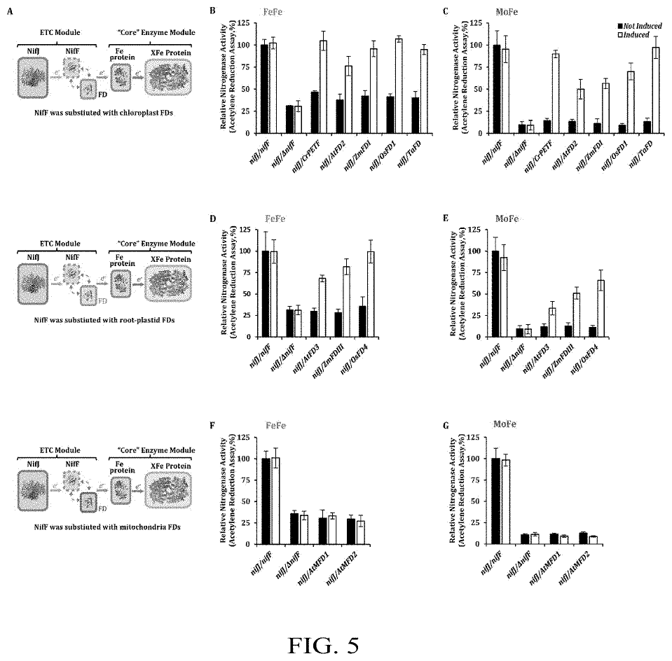

The flavodoxin encoded by NifF from the NifJ-NifF module was replaced by the hybrid modules constructed above and the activity of the recombined MoFe, or FeFe systems were analyzed by the method of acetylene reduction^{[27-28]}. The results show that all hybrid ETC modules could restore nitrogenase activities for both the MoFe and FeFe systems, but with different activities. Values greater than 100% were observed for the FeFe nitrogenase system when NifF was replaced with the ferredoxins from As (FdxH), Cr (PETF), or Os (FD1) respectively (see FIG. 5 and Table 2). This result suggests that the AvAnfH protein in the hybrid “minimal” FeFe nitrogenase^{[28]} may prefer ferredoxin, rather than flavodoxin, as electron donor. All the chloroplast ferredoxins derived hybrid modules could restore about ˜100% activities for the FeFe nitrogenase system, except the NifJ-AtFD2 hybrid module, which showed ˜76% activity (FIG. 5B). The NifJ-CrPETF and NifJ-TaFD hybrid modules could restore >90% activities for the MoFe nitrogenase system. The NifJ-AtFD2, NifJ-ZmFDI and NifJ-OsFD1 hybrid modules showed restored activity to <70% (FIG. 5C). All root-plastid ferredoxin derived hybrid modules showed lower nitrogenase activities when compared with the chloroplast ferredoxin derived modules from the same organism (FIG. 5B-5E).

TABLE 2
The activities of the FeFe or MoFe nitrogenase system after NifF
was replaced by ferredoxins from different plant plastids

| | | | | Relative nitrogenase activity, | |
|---|---|---|---|---|---|
| | | | Redox | % acetylene reduction^{a} | |
| Organism | Location | Genes | potential | FeFe | MoFe |
| Ko | — | nifF | −412 mV(2) | 100 ± 6 | 100 ± 15 |
| | — | ΔnifF | — | 31 ± 6 | 10 ± 3 |
| As | Heterocyst | fdxH | −351 mV(3) | 153 ± 21 | 100 ± 6 |
| | Vegetative | petF | −384 mV(3) | 110 ± 14 | 81 ± 11 |
| Cr | Chloroplast | PETF | −398 mV(4) | 105 ± 11 | 90 ± 5 |
| | Chloroplast | FDX2 | −321 mV(4) | 92 ± 4 | 87 ± 4 |
| Zm | Chloroplast | FDI | −423 mV(5) | 96 ± 9 | 56 ± 6 |
| | Chloroplast | FDII | −406 mV(5) | 75 ± 7 | 50 ± 7 |
| | Root plastid | FDIII | −321 mV(6) | 82 ± 9 | 51 ± 7 |
| At | Chloroplast | FD1 | −425 mV(7) | 59 ± 6 | 36 ± 6 |
| | Chloroplast | FD2 | −433 mV(7) | 76 ± 11 | 50 ± 11 |
| | Root plastid | FD3 | −337 mV(7) | 68 ± 4 | 34 ± 8 |

^{a}Activities of the FeFe or MoFe nitrogenase systems carrying the NifJ-NifF module are represent as 100%. Data presented are mean values based on at least three independent experiments.

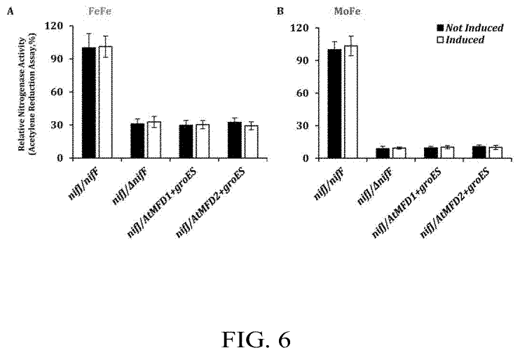

As mitochondria are another potential location for nitrogenase in the plant, the capability of mitochondrial ferredoxins in supporting nitrogen fixation was also investigated in E. coli. The same strategy was used to clone the mitochondria ferredoxin coding genes as described in the former part of this section. When mitochondria ferredoxin derived hybrid ETC modules (NifJ-AtMFD1 or NifJ-AtMFD2) were introduced into E. coli, no restoration of the nitrogenase activities were observed (FIGS. 5F and 5G). Further, in order to exclude the effect of GroESL proteins on proper and efficient folding of the mitochondrial ferredoxins in E. coli, as indicated by Picciocchi et al.^{[26]}, a high-copy plasmid carrying the GroESL encoding genes was co-transformed with a MoFe or FeFe nitrogenase system carrying either the NifJ-AtMFD1 or NifJ-AtMFD2 hybrid modules. Similar negative results were finally obtained (FIG. 6). Simultaneously, phylogenetic analysis also showed that the mitochondrial ferredoxins do not have similar evolutionary relationships with any electron donors of nitrogenase. Overall, these results suggest that the mitochondria ferredoxins cannot couple with the NifJ protein to form functional ETC modules capable of providing electrons for the nitrogenase systems.

### Example 2: Study of Electron Supply of Hybrid ETC Modules Consisting of Plant-Type FNR and KoNifF, AsFdxH and AsFdxB, Respectively, to Nitrogenase System

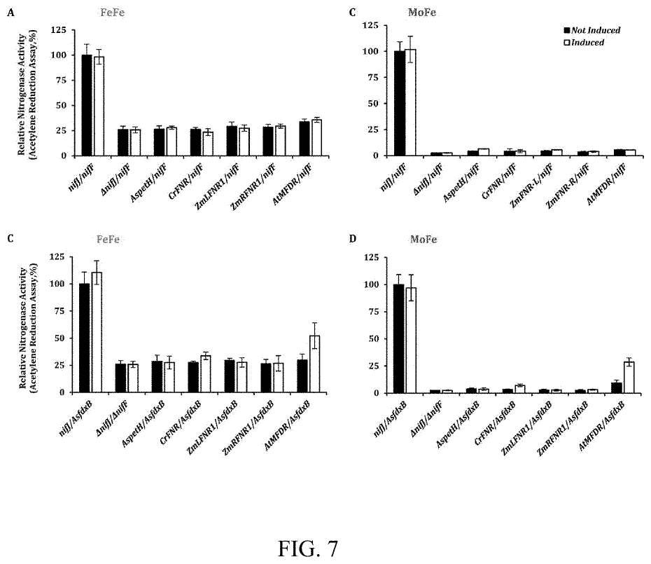

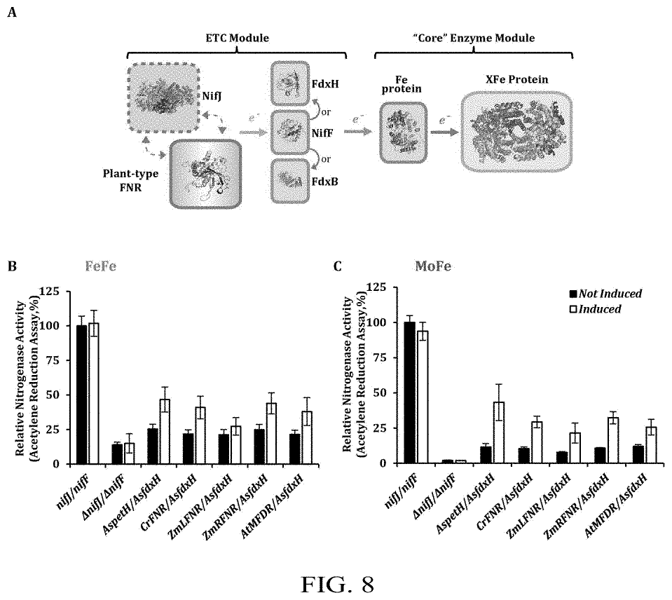

In plants, three different types of the FNRs existing in different organelles are identified. All of these FNRs function to mediate electron transfer between the ferredoxins and NADPH^{[23, 25, 26]}. To investigate if hybrid ETC modules consisting of the plant-type FNRs and electron donors (KoNifF, AsFdxH and AsFdxB) for nitrogenase could mediate electron transfer to nitrogenase, coding sequences of FNRs from the chloroplast or root-plastid of Cr, Zm, or MFDR from mitochondria of At were selected for testing. These hybrid modules were transformed into the E. coli, and their activities were assayed with acetylene reduction method. The results showed that none of the hybrid ETC modules consisting of the plant-type FNRs and the NifF could restore acetylene reduction activity for either the MoFe or FeFe nitrogenase systems; AsFdxB can form functional ETC module to support both of MoFe and FeFe nitrogenases activities only when it is coupled with the MFDR from mitochondria (FIG. 7); all hybrid modules formed with the plant-type FNRs and AsFdxH could restore nitrogenase activity for both of the MoFe and FeFe nitrogenase systems (FIG. 8).

### Example 3: The Intact ETC Modules from the Chloroplast and Root-Plastid Support Nitrogenase Activity

After verifying the function of the hybrid modules as the electron supplier for the nitrogenase systems, further experiments were carried out to investigate whether the intact ETC modules, consisting of FNRs and their cognate ferredoxins from plant organelles, could support the nitrogenase activity. By combining the P_{tac} controlled FNRs with P_{LtetO-1} controlled ferredoxins (details are provided in Materials and Methods), two intact chloroplast ETC modules, CrFNR-PETF and ZmLFNR-FDI; one intact root-plastid ETC module ZmRFNR-FDIII; and one intact mitochondria ETC module AtMFDR-MFD were constructed. As it is known that the AsPetH-FdxH module can support nitrogen fixation in its original host, this module was used as a control.

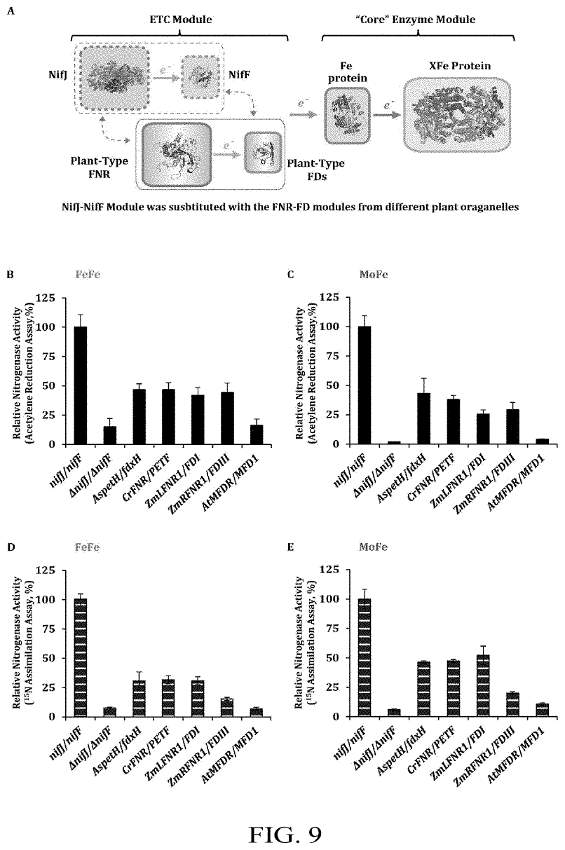

When these intact ETC modules were used to replace the NifJ-NifF modules of either the MoFe or the “minimal” FeFe nitrogenase system respectively, their ability to support nitrogenase activities were assayed with both the acetylene reduction and ¹⁵N assimilation methods. The results showed that with the exception of the AtMFDR-MFD module from mitochondria, all other ETC modules can support acetylene reduction activities and ¹⁵N assimilation activities for both MoFe and FeFe nitrogenases (FIG. 9); no restoration of activity was observed, when either of the two components from each of the modules was expressed individually (FIG. S6), indicating that for the functionality of each plant intact ETC module, both components have to be present.

For the FeFe nitrogenase system, the chloroplast modules, CrFNR-PETF and ZmLFNR-FDI, showed almost equal amount of restored acetylene reduction activities (˜45%) and ¹⁵N assimilation activities (>30%) as that with the AsPetH/FdxH (FIGS. 5B and D). Similar results were obtained for the MoFe nitrogenase system, chloroplast modules CrFNR-PETF and ZmLFNR-FDI, and AsPetH/FdxH module each restored ˜46% of ¹⁵N assimilation activities (FIG. 9E). The ZmRFNR-FDIII module from the root-plastid only restored lower activities for both MoFe and FeFe nitrogenases, compared with chloroplast module from the same organism (FIGS. 9B, C, D and E). Weakly increased activities were observed from the the MoFe nitrogenase system carrying AtMFDR-MFD module (11% ¹⁵N assimilation activity) when compared with the NifJ-NifF deficient negative control (6% ¹⁵N assimilation activity) in the MoFe nitrogenase system (FIG. 9E). But this phenotype was not observed in the FeFe nitrogenase system (FIG. 9D). As multiple-copies of ferredoxins exist in E. coli, we believe that the enhanced activity of MoFe nitrogenase system carrying the AtMFDR-MFD module may be contributed by hybrid modules formed with AtMFDR and the ferredoxins from E. coli. For the FeFe nitrogenase system, such effect may be masked by high background activities due to its high background activities (FIG. 9D).

Taken together, above results demonstrate that the intact ETCs modules from plastids (including chloroplast and root-plastid), but not from mitochondria, are capable of providing the electron and reducing power required to reduce nitrogen for the nitrogenase system.

### REFERENCES

1. Xu G, Fan X, & Miller A J (2012) Plant Nitrogen Assimilation and Use Efficiency. Annual Review of Plant Biology 63 (1): 153-182.
2. Galloway J N, et al. (2008) Transformation of the Nitrogen Cycle: Recent Trends, Questions, and Potential Solutions. Science 320(5878):889-892.
3. Tilman D, Balzer C, Hill J, & Befort B L (2011) Global food demand and the sustainable intensification of agriculture. Proceedings of the National Academy of Sciences 108(50):20260-20264.
4. Canfield D E, Glazer A N, & Falkowski P G (2010) The Evolution and Future of Earth's Nitrogen Cycle. Science 330(6001):192-196.
5. Schindelin H, Kisker C, Schlessman J L, Howard J B, & Rees D C (1997) Structure of ADP.AIF4—stabilized nitrogenase complex and its implications for signal transduction. Nature 387(6631):370-376.
6. Tezcan F A, et al. (2005) Nitrogenase Complexes: Multiple Docking Sites for a Nucleotide Switch Protein. Science 309(5739):1377-1380.
7. Eady R R, Richardson T H, Miller R W, Hawkins M, & Lowe D J (1988) The vanadium nitrogenase of Azotobacter chroococcum. Purification and properties of the Fe protein. Biochemical Journal 256(1):189-196.
8. Schneider K, Gollan U, Drottboom M, Selsemeier-Voigt S, & Müller A (1997) Comparative Biochemical Characterization of the Iron-Only Nitrogenase and the Molybdenum Nitrogenase from Rhodobacter Capsulatus. European Journal of Biochemistry 244(3):789-800.
9. Halbleib C M & Ludden P W (2000) Regulation of Biological Nitrogen Fixation. The Journal of Nutrition 130(5):1081-1084.
10. Duval S, et al. (2013) Electron transfer precedes ATP hydrolysis during nitrogenase catalysis. Proceedings of the National Academy of Sciences 110(41):16414-16419.
11. Hageman R V & Burris R H (1978) Nitrogenase and nitrogenase reductase associate and dissociate with each catalytic cycle. Proceedings of the National Academy of Sciences 75(6):2699-2702.
12. Hu Y, et al. (2006) Nitrogenase Fe protein: A molybdate/homocitrate insertase. Proceedings of the National Academy of Sciences 103(46): 17125-17130.
13. Shah V K, Stacey G, & Brill W J (1983) Electron transport to nitrogenase. Purification and characterization of pyruvate:flavodoxin oxidoreductase. The nifJ gene product. Journal of Biological Chemistry 258(19):12064-12068.
14. Schmitz O, Gurke J, & Bothe H (2001) Molecular evidence for the aerobic expression of nifJ, encoding pyruvate:ferredoxin oxidoreductase, in cyanobacteria. FEMS Microbiology Letters 195(1):97-102.
15. Yakunin A F & Hallenbeck P C (1998) Purification and characterization of pyruvate oxidoreductase from the photosynthetic bacterium Rhodobacter capsulatus. Biochimica et Biophysica Acta (BBA)—Bioenergetics 1409(1):39-49.
16. Schmehl M, et al. (1993) Identification of a new class of nitrogen fixation genes in
Rhodobacter capsalatus: a putative membrane complex involved in electron transport to nitrogenase. Mol Gen Genet 241(5-6):602-615.
17. Neuer G & Bothe H (1985) Electron donation to nitrogenase in heterocysts of cyanobacteria. Arch. Microbiol. 143 (2): 185-191.
18. Cheng Q, Day A, Dowson-Day M, Shen G-F, & Dixon R (2005) The Klebsiella pneumoniae nitrogenase Fe protein gene (nifH) functionally substitutes for the chlL gene in Chlamydomonas reinhardtii. Biochemical and Biophysical Research Communications 329(3):966-975.
19. Ivleva N B, Groat J, Staub J M, & Stephens M (2016) Expression of Active Subunit of Nitrogenase via Integration into Plant Organelle Genome. PLoS ONE 11(8):e0160951.
20. Lopez-Torrejon G, et al. (2016) Expression of a functional oxygen-labile nitrogenase component in the mitochondrial matrix of aerobically grown yeast. Nat Commun 7.
21. Hanke G U Y & Mulo P (2013) Plant type ferredoxins and ferredoxin-dependent metabolism. Plant, Cell & Environment 36(6):1071-1084.
22. Fukuyama K (2004) Structure and Function of Plant-Type Ferredoxins. Photosynth Res 81(3):289-301.
23. Kurisu G, et al. (2001) Structure of the electron transfer complex between ferredoxin and ferredoxin-NADP+reductase. Nat Struct Mol Biol 8(2):117-121.
24. Knaff D B & Hirasawa M (1991) Ferredoxin-dependent chloroplast enzymes. Biochimica et Biophysica Acta (BBA)—Bioenergetics 1056(2):93-125.
25. Green L S, et al. (1991) Ferredoxin and Ferredoxin-NADP Reductase from Photosynthetic and Nonphotosynthetic Tissues of Tomato. Plant Physiology 96(4):1207-1213.
26. Picciocchi A, Douce R, & Alban C (2003) The Plant Biotin Synthase Reaction: IDENTIFICATION AND CHARACTERIZATION OF ESSENTIAL
MITOCHONDRIAL ACCESSORY PROTEIN COMPONENTS. Journal of Biological Chemistry 278(27):24966-24975.
27. Wang X, et al. (2013) Using Synthetic Biology to Distinguish and Overcome Regulatory and Functional Barriers Related to Nitrogen Fixation. PLoS ONE 8(7):e68677.
28. Yang J, Xie X, Wang X, Dixon R, & Wang Y-P (2014) Reconstruction and minimal gene requirements for the alternative iron-only nitrogenase in Escherichia coli. Proceedings of the National Academy of Sciences of the United States of America 111(35):E3718-E3725.
29. Wang L, et al. (2013) A Minimal Nitrogen Fixation Gene Cluster from Paenibacillus sp. WLY78 Enables Expression of Active Nitrogenase in Escherichia coli. PLoS Genetics 9(10):e1003865.
30. Li X-X, Liu Q, Liu X-M, Shi H-W, & Chen S-F (2016) Using synthetic biology to increase nitrogenase activity. Microbial Cell Factories 15:43.
31. Gatti-Lafranconi P, Dijkman W P, Devenish S R, & Hollfelder F (2013) A single mutation in the core domain of the lac repressor reduces leakiness. Microbial Cell Factories 12(1):67.
32. F. C. Cannonl RADaJRP (1976) Derivation and Properties of F-prime Factors in Escherichia coliCarring Nitrogen Fixation Genes from Klebsiella pneumoniae. Microbiology 93(1):111-125.
33. J. P. Montoya M V, P. Kahler, and D. G. Capone (1996) A Simple, High-Precision, High-Sensitivity Tracer Assay for N(2) Fixation. Appl Environ Microbiol 62(3):986-993.
34. Masepohl B, Schölisch K, Gorlitz K, Kutzki C, & Böhme H (1997) The heterocyst-specific fdxH gene product of the cyanobacterium Anabaena sp. PCC 7120 is important but not essential for nitrogen fixation. Mol Gen Genet 253(6):770-776.

1. An electron transport chain (ETC) module for a biological nitrogen fixation system, comprising an NifJ protein and an NifF protein.

2. The ETC module of claim 1, wherein the nitrogen fixation system is a MoFe nitrogenase system and an FeFe nitrogenase system.

3. The ETC module of claim 1, wherein the NifJ protein and the NifF protein are substituted individually or substituted simultaneously by corresponding proteins from eukaryotic organelles, thereby forming hybrid or intact ETC modules.

4. The ETC module of claim 3, wherein the eukaryotic organism is a plant, and the organelle is a plastid or mitochondria.

5. The ETC module of claim 3, wherein the hybrid ETC module is formed by replacing the NifF protein in the ETC module consisting of the NifJ and the NifF with a ferredoxin from a plant plastid.

6. The ETC module of claim 5, wherein the plastid is a chloroplast or a root-plastid, preferably a chloroplast.

7. The ETC module of claim 3, wherein the hybrid ETC module is formed by replacing the NifJ protein in the ETC module consisting of the NifJ and the NifF with a plant-type Ferredoxin-NADPH reductase (FNR) from a plant plastid and mitochondria.

8. The ETC module of claim 7, wherein the plastid is a chloroplast or a root-plastid.

9. The ETC module of claim 3, wherein the hybrid ETC module is composed of an NADPH-dependent adrenodoxin oxidoreductase (MFDR) from a plant mitochondria and an Anabaena FdxB.

10. The ETC module of claim 3, wherein the intact ETC module is composed of an FNR from a target plant organelle and Ferredoxin proteins.

11. The ETC module of claim 10, wherein the target plant organelle is a chloroplast or a root-plastid.

12. Use of the ETC module of claim 1 in biological nitrogen fixation.

13. Use of the ETC module of claim 6 in biological nitrogen fixation.

14. Use of the ETC module of claim 7 in biological nitrogen fixation.
